(19) United States
(12) Patent Application Publication
(10) Pub. No.: US 2025/0073531 A1
(43) Pub. Date: Mar. 6, 2025

(54) METHOD FOR PREDICTING AEROBIC THRESHOLD AND ANAEROBIC THRESHOLD AND ESTABLISHING THE OPTIMAL AEROBIC TRAINING ZONE

(71) Applicant: SAMSUNG ELETRÔNICA DA AMAZÔNIA LTDA., CAMPINAS (BR)

(72) Inventors: LUIS ANTONIO DE SOUZA JÚNIOR, CAMPINAS (BR); THOMAS BELTRAME, CAMPINAS (BR); GIULIANO ROBERTO PINHEIRO, CAMPINAS (BR); GUSTAVO OLIVEIRA DE SIQUEIRA, CAMPINAS (BR); WILLIAN PULIDO BENEDUCCI, CAMPINAS (BR); INTAEK OH, SEONGNAM-SI (KR); DONGHYUN LEE, SEOUL (KR)

(73) Assignee: SAMSUNG ELETRÔNICA DA AMAZÔNIA LTDA., CAMPINAS (BR)

(21) Appl. No.: 18/517,674

(22) Filed: Nov. 22, 2023

(30) Foreign Application Priority Data
Sep. 4, 2023 (BR) 10 2023 017962 2

(51) Int. Cl. A63B 24/00

(52) U.S. Cl. CPC A63B 24/0062; A63B 2024/0065; A63B 2230/015; A63B 2230/045

(57) ABSTRACT

Predict aerobic threshold and anaerobic threshold from data obtained from wearable devices during unsupervised exercise sessions, typical of daily exercise routines. These physiological-based predictions from wearable devices have the potential to improve the general health of a wider audience that may request individualized fitness guidance to train at the OATZ. In short, anthropometric and physiological features, such as gender and heart rate, are collected using wearable devices and used to feed machine learning models to predict the AT and AnT that can be used to define the upper and lower bound, respectively, of the optimal aerobic training zone.

### CROSS-REFERENCE TO RELATED APPLICATION(S)

This application is based on and claims priority under 35 U.S.C. § 119 to Brazilian Patent Application No. BR 102023017962-2, filed on Sep. 4, 2023, in the Brazilian Intellectual Property Office, the disclosure of which is incorporated by reference herein in its entirety.

### TECHNICAL FIELD

Physiological thresholds are crucial for establishing personalized physical activity training with better clinical outcomes, as well as for the evaluation of the health-related physical fitness level. The aerobic and anaerobic thresholds (AT and AnT, respectively) are the most common thresholds used for these purposes because they define the optimal aerobic training zone (OATZ) and are influenced by the fitness status.

AT and AnT are usually obtained in laboratory settings by cumbersome equipment during maximal exercise protocols. Hence, the present invention aims to predict AT and AnT from data obtained from wristbands during unsupervised exercise sessions, typical daily of exercise routines. These physiological-based predictions from wearable devices have the potential to improve the general health of a wider audience that may request individualized fitness guidance to train at the OATZ. In short, anthropometric and physiological features, such as gender and heart rate, are collected using wearable devices and used to feed machine learning models to predict the AT and AnT that can be used to define the upper and lower bound, respectively, of the OATZ. The OATZ is delivered to smartwatch users to help them improve their general health by personalized physiological-based training zone indication.

The transition moment when the anaerobic systems start to play a major role in energetic supply is one of the most valuable human physiological events for personalized endurance training and fitness level assessment. Several methods to identify this transition were proposed, including the consideration of the following variables: first and second ventilatory thresholds (VT1 and VT2), lactate threshold (LT), and the heart rate variability deflection point. VT1 is also known as the gas exchange threshold (GET) while the VT2 is also known as respiratory compensation point (RCP). These methods are employed for monitoring training schedules, fitness progress, and sports performance evaluation.

Across the twentieth century, the definition of aerobic and anaerobic thresholds (AT and AnT, respectively) have been studied extensively and updated. The AT indicator, often expressed through the LT or the VT1, correlates with the moment in which the pulmonary ventilation is influenced the most by the blood lactate buffering (increased carbon dioxide output, VCO₂) than the proper total O₂ muscular cell consumption (VO₂). The AnT, usually described by the VT2, defines the moment in which the exercise-induced pulmonary hyperventilation is more evident due to the metabolic acidosis that occurs by the blood lactate accumulation. Both indicators (AT and AnT) can be employed to determine the user's fitness level and to define a personalized optimal aerobic training zone (OATZ).

The AT and AnT, as moments in time, can be expressed in any useful unit at a specific instant (i.e., beats per minute—bpm, for example), allowing the real-time control of the OATZ for a better and individualized endurance training. Hence, the main objective of the present invention is to evaluate a large set of physiological features acquired from smartwatch sensors and observed during daily activities. Therefore, machine learning (ML) techniques are applied to predict AT and AnT indicators (expressed as heart rate—bpm).

Moreover, the personalized OATZ is also established based on these indicators. Finally, the method for predicting AT, AnT, and OATZ is encoded into smartwatches, which provides the parameter for feeding machine learning algorithms. Therefore, a personalized experience is proposed by calculating OATZ based on AT and AnT, encouraging ordinary users to improve their training routines.

The paper entitled “Estimation of lactate threshold with machine learning techniques in recreational runners”, published in February 2018, by Extegarai, et al., proposes an intelligent system capable of estimating the lactate threshold of recreational athletes participating in endurance running sports. That work is based on a machine learning system that models the lactate evolution using recurrent neural networks and includes the proposal of standardization of the temporal axis to homogenize different length time-series (heart rate and rate of perceived exertion observations) as well as a modification of the stratified sampling method for the train-test set splitting of the series. In this sense, the present application aims to provide a similar solution of calculating AT and AnT indexes but in a real file approach in which personalized trainings can be suggested to users using the prediction of such fitness indexes.

The paper entitled “Determination of athletes' anaerobic threshold using machine learning methods”, published in March 2022, by Chikov, et al., discloses the development of a model to determine athletes' anaerobic threshold based on observations of their physiological indicators (heart rate, respiratory minute volume, and oxygen consumption). The authors collected the data using Quark CPET treadmill to failure. Linear Regression, Random Forest Regression, Gradient Boosting, and Support Vector Regression (SVR) from the Scikit-learn library were used to determine the physiological parameters of energy supply at the anaerobic threshold. Hence, such a model determines the values and provides a Local-Interpretable Model-Agonistc approach by determining the quantitative measurements of physiological indicators at the anaerobic threshold moment. However, the solution proposed by the present invention delivers a personalized estimation of AT and AnT using data provided by wearable devices. This way, not only this solution proposes a personalized training approach, but also targets the real-time training guidance, prediction the fitness indexes to maintain user's track during ordinary trainings.

The patent document US 20220199244 A1, entitled “Method for Predicting Maximal Oxygen Uptake in Wearable Devices”, published on Jun. 23, 2022, by SAMSUNG ELETRONICA DA AMAZONIA LTDA., introduces a method for predicting maximal oxygen uptake (VO2Max) in wearable devices with memory restriction using an ensemble of machine learning algorithms. This method is related to well-being, healthcare, and artificial intelligence areas, including techniques to predict maximal oxygen uptake in running sessions at different paces using wearable devices with memory restriction. The proposed method requires less than 5 KB of memory to run on a smart wearable device. More specifically, equipped with the user's profile data (age, gender, height, and weight), a set of heart rate, and speed readings of an outdoor activity session, the regressor can estimate VO2Max. This patent is correlated with the present invention once the same wearable devices, sensors, and personal information are gathered for prediction purposes. The VO2Max describes one fitness indicator and, in a logical progression of it, the present invention aims to extend it by displaying AT, AnT, and the range of optimal training zone (OATZ), leading users to improve all their fitness routines and indicatives.

The patent document U.S. Pat. No. 8,784,115 B1, entitled “Athletic training optimization”, published on Jul. 22, 2014, by CHUANG THOMAS CHU-SHAN, comprises a complex workflow to follow athletic activities composed of a processor, a user interface, a heart rate monitor, a navigation system, memory storage, and an application. The heart rate monitor has an interface configured to collect heart rate data. The navigation system is responsible for recording the user location and speed data. The memory storage comprises executable instructions for the processor. At last, the application assists in the training intensities definition and updates. The present invention, in counterpart, recommends optimal training zones based on fitness indicators AT and AnT predicted from wearable data.

The patent document U.S. Pat. No. 11,412,979 B2, entitled “Apparatus and method for estimating anaerobic threshold”, published on Aug. 16, 2022, by SAMSUNG ELECTRONICS CO. LTD., depicts an equipment solution for estimating anaerobic threshold (AnT), including a heart rate detector. The anaerobic threshold estimator is configured based on the heart rate changes. The method configuration determines the heart rate flex point based on a predetermined exercise. This exercise aims to find the stable state of the heart rate activity to contrast with its steady state. More than just estimating the anaerobic threshold (AnT), the present invention predicts the aerobic threshold (AT) presented in the literature from physiological reasoning, the range between such fitness markers stands for the optimal aerobic training zone. Thus, wearable devices will encode the method described herein, and will inform and suggest users about optimal training zones.

The patent document US 2017143262 A1, entitled “Systems, methods, computer program products, and apparatus for detecting exercise intervals, analyzing anaerobic exercise periods, and analyzing training effects”, published on May 25, 2017, by FIRSTBEAT TECH OY, introduces a method for determining the anaerobic training effect (namely anaerobic phase) of a user in an exercise based on heart-rate monitoring and its high-intensity period (as the exercise interval if its interval-likeness value is higher than a predetermined threshold value, and represented by at least one derivative of the intensity) based on (i) the amount of intensity increase during the high intensity period; (ii) the duration of the high-intensity period; and (iii) an external workload during the high-intensity period. This document's proposal differs from the present application, as while the aim of the present application is to encode exercise information from several types of workouts, the aforementioned solution delivers one or some anaerobic training effects based on only heart-rate monitoring, heat-rate surveillance during training and other not-well defined physiological features. Furthermore, the present invention uses machine-learning generalization, where the devices contain pre-trained models to proper generalize AT and AnT based on experimentation. However, the solution proposed in the document calculate several of its input indexes based on literature's fixed formulations, such as anaerobic multiplier, implying in a less personalized-driven method.

### SUMMARY

A method of predicting aerobic threshold (AT) and anaerobic threshold (AnT) and establishing an optimal aerobic training zone (OATZ). The method comprises receiving personal data; feeding a regression-based model with the personal data to generate a first intermediate prediction (rboutput); capturing temporal signals (x) including heart rate and speed readings; splitting the temporal signals into n-second time windows; processing the n-second time windows. The processing includes: identifying points where heart rate and speed signals satisfy a stability condition; extracting features of stable temporal signals (xₜ) through a neural networks-based model; feeding a MLP machine learning model with the features of stable temporal signals to generate a second intermediate prediction (mlpoutput); combining intermediate predictions performed by machine learning models and regression-based estimators, based on a pre-defined threshold tao to a range of tolerance values for each index; ATtao(f)=ATMLP(f′)+ATregbas(f″)/nestimators, AnTtao(f)=AnTMLP(f′)+AnTregbas(f″)/nestimators, where tao is related to post-processed AT and AnT final predictions, MLP refers to MLP-model predictions, regbas refers to regression-based model predictions, nestimators is a number of models employed for AT and AnT predictions, f is a descriptor composed of temporal and anthropometric features, and f′, f″ are subsets of a whole set of features of f, with anthropometric (f′) and temporal features (f″) features, respectively; and defining the OATZ as a range [AT, AnT], in beats per minute and pace.

### BRIEF DESCRIPTION OF THE DRAWINGS

The objectives and advantages of the current invention will become clearer through the following detailed description of the example and non-limitative drawings presented at the end of this document.

FIG. 1 relates to an overview of the proposed solution according to an exemplary embodiment of the present invention.

FIG. 2 presents details about the implementation of the present invention according to an exemplary embodiment of the present invention.

FIG. 3 illustrates an exemplary embodiment of the present invention.

FIG. 4 depicts details according to an exemplary embodiment of the present invention.

FIG. 5 depicts the Gait Enhancing and Motivation System (GEMS) device according to an exemplary embodiment of the present invention.

FIGS. 6A and 6B show the wearable displays when running the proposed solution in a hypothetical practical scenario according to an exemplary embodiment of the present invention.

### DETAILED DESCRIPTION

The technical problem related to AT/AnT/OATZ prediction is related to which ML-based model to be employed for the correct calculation of such fitness indexes. The proposed regression task is highly suitable for several linear-based and neural networks-based models. The main goal of this proposal is to develop a hybrid model to perform the prediction of AT and AnT. An optimal prediction framework is designed, evaluating from regressors to neural networks. Also, to enhance the predictions, hyperparameter fine-tuning and model ensemble are also implemented.

From observations, the predictions based on anthropometric features fit regression-model generalization better than neural networks. The opposite occurs with measures and gathered features. Hence, two different models, further ensembled, to predict AT and AnT, delivering high fidelity on wearable devices. First, the anthropometric features feed linear-based models, such as Decision Tree Regressor and Random Forest Regressor. Meanwhile, sensor-measured ones are used in shallow neural nets (Multilayer Perceptron architectures) for regression purposes. Ultimately, the final prediction of AT and AnT relies on both models, one focused on anthropometric data and the other based on sensor-measured data. It is imperative to highlight that the correct estimation of AT and AnT using the same model is challenging. Due to that, a model for each index is proposed, which is further composed of inner models for processing specific features.

Concerning the neural networks-based step in the prediction, another challenge arises: the time-series data must be processed to provide the sample's feature vector. To cope with this task, time-specialized neural models are utilized, such as Long-Short Term Memory (LSTM), producing highly discriminative temporal features.

The proposed technique has several advantages for embedded applications in wearable devices, such as smartwatches. In this sense, the present invention can obtain a reasonably accurate prediction of AT and AnT using only available data from ordinary smartwatch sensors: profile data, heart rate based on photoplethysmogram sensor speed (PPG), computed using accelerometer sensor or GPS libraries, and step frequency estimated using the accelerometer. Another advantage is the recommendation of optimal training zones, which can help thousands of people to improve their fitness indicators in a highly personalized solution inside their smartwatches, for example. Besides, the estimation of AT/AnT indexes during ordinary exercises, such as walking or free-running exercises, is something not trivial, once these indicators are mostly detected during maximal (intense) exercises.

Hence, the estimation of AT and AnT indexes promotes a logical extension to personalized solutions currently proposed, increasing the personalization level delivered to every single wearable user by delivering ranges of intensity to optimize their trainings and positive outcomes.

In view of that, the present invention contemplates the following procedures: (i) the estimation of AT and AnT fitness indicators; (ii) the recommendation for personalized optimal aerobic training zones (OATZ); (iii) unreliable sensor data preprocessing; and (iv) the estimation procedure adapted to users target scenario.

The first aim of the present invention is to calculate the OATZ based on two physiological indicators, AT and AnT. In this sense, reference values of AT and AnT are computed using collected physiological attributes (heart rate, speed acceleration, step frequency, body mass index, maximum oxygen uptake). Further, using machine learning techniques, a predictor of both AT and AnT is proposed based on the correlation between sensor-measured features and anthropometric features (gender, age, height, weight).

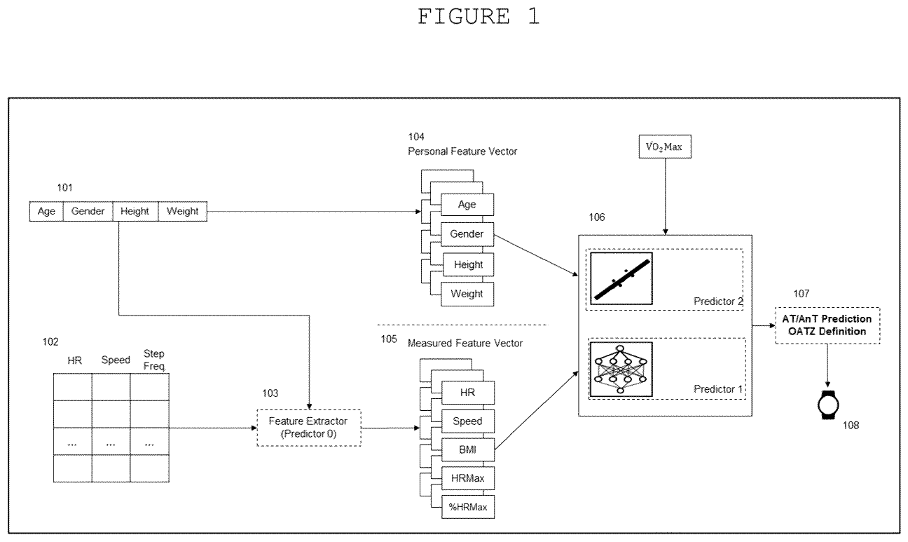

FIG. 1 depicts a brief overview of the scheme proposed herein. Firstly, information from the user profile (101) is including captured, age (in years), gender (male/female), height (in centimeters), and weight (in kilograms). Secondly, temporal inputs (102) composed of readings of, for instance, heart rate (in bpm), speed (in kilometers per hour), and step frequency (steps per second). The measures can be made through sensors, such as: GPS or pedometer, accelerometer, not limited to just these, collected during N seconds and processed by a feature extraction module (103). The feature extraction module is responsible for temporal-related feature stabilization and feature engineering, which includes:
Maximum heart rate (HRMax): the theoretical maximum heart rate, estimated based on age.
Percentage of maximum heart rate (% HRmax): heart rate proportion w.r.t maximum heart rate computed.
Body-Mass-Index (BMI): physiological index calculated based on user height and weight.

Finally, the anthropometric data (personal features vector (104)) and sensor-measured data (measured feature vector (105)) feed two different machine learning architectures, each responsible for a set of features (106), including the last VO2Max estimation.

These feature vectors are used as input for an ensemble composed of two machine learning models, each tailored for a specific scenario:
A MLP regressor for sensor-measured features; and
A regressor-based model for anthropometric features.

The prediction of AT and AnT values and OATZ range (107) relies on both machine learning models described in the prediction module (106). Finally, with the OATZ information in the smartwatch (108), a user can monitor the intensity of physical exercises to achieve improvements and possible adaptations for the training, focusing on enhancing the fitness outcome from it.

Hence, the establishment of OATZ relies on:
Machine learning models based on (i) regression techniques and (ii) neural networks to cope with the generalization and prediction of aerobic and anaerobic thresholds. These models should respect the following constraints:
A) The designed model must cope with both predictions, AT and AnT indicators.
B) All features should be preprocessed to obtain suitable characteristics for predicting AT and AnT, respectively.

The majority of features to feed the machine learning models must be defined, including anthropometric and sensor-measured:
I) Anthropometric features are related to non-measured features and previously inputted by users. This set comprises gender, age, height, weight, body mass index, and maximum heart rate.
II) Sensor-measured features are related to data using reference equipment (Quark CPET, K5, Polar Team Pro) for the training and data from wearables for prediction. This set comprises VO2Max, heart rate, speed, and step frequency. The set of sensor features is not restricted to the aforementioned ones, while extra features from devices can compose this set.

Sensor-measures features are related to exercises and are composed of several observations over time, resulting in time-series. Due to that, it is imperative to define a way to deal with time-series features, employing techniques to encode their information, such as long-short term memory nets or intermediate assessment and prediction based on sliding window evaluation, for instance. The task of handling time-series significantly increases the correct prediction rates, configuring an aspect to be highlighted and observed.

The proper definition of OATZ, based on AT and AnT predictions, can be displayed on the smartwatch as a solution to keep users training inside the optimal aerobic zone.

The method presented herein establishes stabilization mechanisms based on previous predictions considering physiological-anthropometric correlation to AT and AnT indicators.

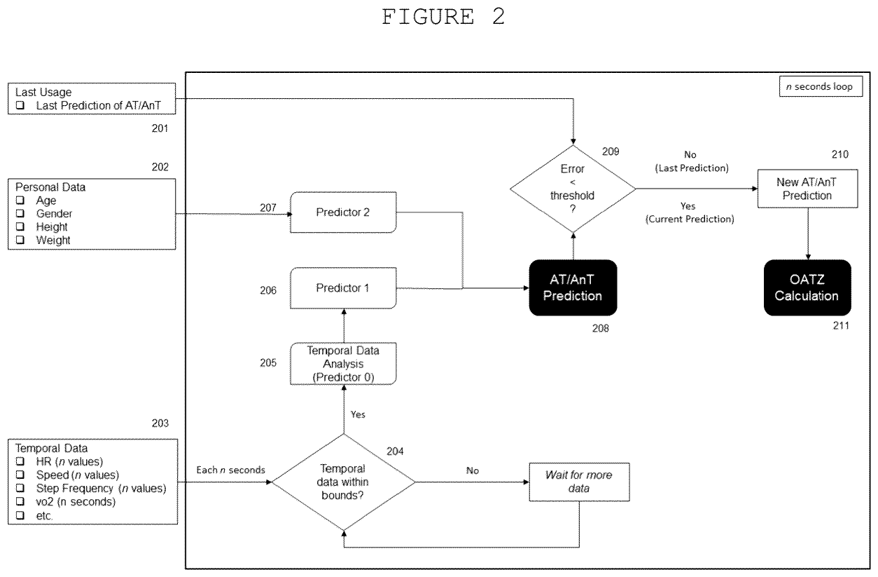

FIG. 2 explains in more detail the prediction pipeline of the proposed method. Firstly, capturing information from user profile (202), including age (in years), gender (male/female), height (in centimeters), and weight (in kilograms). Secondly, capturing the temporal inputs (203) composed of readings of heart rate (in bpm), speed (in kilometers per hour), step frequency (in steps per second), and record the n seconds time-series (204), which are processed by a temporal data analysis module (205). The time-series feature extraction module (205) is responsible for processing the time series data using machine learning techniques to optimally describe the measured data. Finally, the profile data (202) and the time series data (205) feed independent machine learning models (206 and 207, respectively), one for each kind of feature, to predict the fitness measures (208). Moreover, there is a post-processing step (209) to decide whether to output the new prediction (210) or to repeat the last stable value (201), depending on whether the time-series deviation exceeds a predetermined threshold. The OATZ (211) is displayed after the previous processes highlighted in FIG. 2.

The proposed method is composed of the fusion of two different models for the prediction task and the application of a third model for dealing with time-series data that feed one of the models. Each of the three models is fine-tuned to a specific target (temporal scenario feature extraction, demographic data, and sensor data). Beyond the model fusion, the approach introduces pre-processing and post-processing steps tailored to each scenario to avoid the influence of bad quality input data and leverage past predictions to mitigate prediction failures in practical terms. The compared results are detailed below:

### Time-Series Model (Predictor 0)

This model requires the composition of a feature vector based on the evaluation of time-series data to provide accurate predictions of AT and AnT. Trivial handcrafted features obtained over the data (mean and standard deviation) do not help the models to generalize values of AT and AnT. Hence, a time-series data pre-processing is performed to extract temporal features by employing models such as LSTM, and statistical measurements, such as the coefficient of variation.

Given a single time step, the LSTM processing expected is ruled by:

$${f}_{t} = {σ}_{g} ( {W}_{f} \times {x}_{t} + {U}_{f} \times {h}_{t - 1} + {b}_{f} ) \qquad ( 1 )$$

$${i}_{t} = {σ}_{g} ( {W}_{i} \times {x}_{t} + {U}_{i} \times {h}_{t - 1} + {b}_{i} ) \qquad ( 2 )$$

$${o}_{t} = {σ}_{g} ( {W}_{o} \times {x}_{t} + {U}_{o} \times {h}_{t - 1} + {b}_{o} ) \qquad ( 3 )$$

$${c}_{t}^{′} = {σ}_{c} ( {W}_{c} \times {x}_{t} + {U}_{c} \times {h}_{t - 1} + {b}_{c} ) \qquad ( 4 )$$

$${c}_{t} = {f}_{t} \cdot {c}_{t - 1} + {i}_{t} \cdot {c}_{t}^{′} \qquad ( 5 )$$

$${h}_{t} = {o}_{t} \cdot {σ}_{c} ( {c}_{t} ) \qquad ( 6 )$$

where fₜ is the forget gate, iₜ is the input gate, oₜ is the output gate, cₜ is the cell state, hₜ is the hidden state, σ_{g} means sigmoid, and σ_{c} represents a tanh function.

Given a temporal signal x, the coefficient of variation (CoV) is ruled by:

$$\overline{x} = \frac{{Σ}_{i = 1}^{n} {x}_{i}}{n - 1} ; \qquad ( 7 )$$

$$C o V = \frac{\overline{x}}{\sqrt{\frac{{Σ}_{i = 1}^{n} {( {x}_{i} - \overline{x} )}^{2}}{n - 1}}} . \qquad ( 8 )$$

Hence, for processing the heart rate and speed data, but still ensuring the personalization, a called “stable window” design is used, in which only data that satisfies pre-defined threshold conditions for are considered the workout representation. After such processing, the temporal features are calculated (based on the aforementioned methods), describing not only heart rate and speed nuances, but also their global temporal behavior.

The proposal is to design a model that learns the correct behavior of temporal data, projecting them to a learned embedding space for the modules in sequence. Therefore, given an N×M dimensional input, where N represents the number of observations and M stands for the number of features, the model output is a feature vector that comprises the temporal information in a 1×N-dimensional structure. The target of the prediction model is the corresponding AT or AnT values.

This module is required in the task of AT/AnT prediction, turning the time-series stabilized and robust to collecting and storing errors.

The time-series model (Predictor 0) is trained exclusively on time-series, M-observed features based on wearable sensors.

### Measured Data Prediction (Predictor 1)

This model comprises a MLP model fed by the output of the Predictor 0 model, which computes the first output concerning the fitness indicators. In this sense, Mean Absolute Error (MAE) and Mean Average Percentage Error (MAPE) are used to assess the generalization capability for the training and testing stages.

The MLP model consists of fully-connected layers of neurons arranged in a feedforward fashion, where each neuron is coupled with a possibly nonlinear activation function. Concretely, the intermediate result of each layer can be expressed by an affine transformation A\vec{x}+\vec{b} over the layer's inputs \vec{x}, composed with an activation function (as ReLU(x)=max(0,x)) activations, for instance). The A and \vec{b} components for all layers correspond to the trainable parameters of this machine learning method.

In sequence, a hyperparameter search process is implemented for the MLP, yielding a fully-connected neural network with i hidden layers and j hidden neurons, respectively, batch normalizations and activations all over each layer but the output (regression). As an output if an MLP-based architecture is employed as the temporal data encoding, an intermediate descriptor mlpₒᵤₜₚᵤₜ is calculated.

Moreover, the Predictor 1 model can be trained exclusively on already processed time-series data, output of Predictor 0 model, or with the association of temporal features (from Predictor 0) combined with personal information. Hence, for Predictor 1's inputs, one can have the memory state cₜ of Predictor 0, or the coefficient of variation (CoV) measure, combined or not with user information (gender, age, height and weight).

### Personal Data Prediction (Predictor 2)

The personal data model correlates the AT and AnT values based on anthropometric features, a context in which the users inform the device their profile data. Therefore, this module estimates the fitness indicators based on several regression-based techniques. Based on the Mean Average Percentage Error (MAPE) metric, the model with the lowest error is chosen, composing a linear combination of personal data that better describes the indicators to be predicted. The output of Predictor 2 is described by:

$${rb}_{output} = c + ( rb 0 * G ) + ( rb 1 * A ) + ( rb 2 * W ) + ( rb 3 * H ) \qquad ( 9 )$$

where G refers to gender (0=female and 1=male), A refers to age in years, W refers to weight in Kg, H refers to height in cm, rb0 to rb3 and c referring to regression-based model parameters and bias, respectively, calculated during the training.

To provide a more personalized output, the models' inputs are based on user information combined with temporal features, i.e., heart rate and speed, collected from device sensors. The temporal features are processed in a called “stable window” design, in which only trustworthy data that satisfies pre-defined conditions (stability criteria) are considered for the workout representation: for identifying points where heart rate and speed signals are stable, i.e., 30 seconds of data that satisfies the stability condition (heart rate's standard deviation below a heart rate threshold and speed's standard deviation below a speed threshold), each observation (sampled at 1 Hz) of each signal is compared to a threshold on its standard deviation; calculating the standard deviation of a temporal signal with a moving average X_{A->A+M}, which is an average of the temporal signal with a sliding window of M seconds with a stride of M seconds; to accumulate every stable 30 seconds of temporal data (and compute the final processed-and-cleaned temporal signal), excluding windows that do not satisfies the aforementioned stability criteria, building a new filtered temporal signal that comprises both temporal behavior and outlier-free information:

$${temp}_{signal} = {temp}_{signal} + ( {signal}_{data} \leq {StandardDeviation}_{Threshold} ) , \qquad ( 10 )$$

where temp_{signal} corresponds to heart rate and speed filtered signals, signal_{data} is the raw temporal data and respective StandardDeviation_{Threshold} relates to the corresponding threshold (heart rate or speed) defined for the stable window criteria.

After such processing, the temporal features are calculated based on the coefficient of variation, for instance, describing not only heart rate and speed nuances, but also their global temporal behavior.

Another very important feature incorporated to model's inputs is the VO2Max indicator. The VO2Max index represents the maximum oxygen intake capability, and it is highly correlated to AT and AnT indexes. Considering the progression nature established by the relation AT<<AnT<<HRMax/VO2Max, the restriction implied by VO2Max as an input is necessary to ensure such relation.

Still considering the relation defined by AT<<Ant <<HRMax/VO2Max, these models introduce regularization factors:
I) AT model inputs—user information, hear rate feature, speed feature, VO2Max, AnT; and
II) AnT model inputs—user information, heart rate feature, speed feature, VO2Max, HRMax.

Predictor 2 model is trained exclusively on personal data informed by the users.

### AT/AnT Prediction

Predictors 1 and 2 compose an ensemble module responsible for predicting the final AT/AnT indexes. Each of these predictors are responsible for AT/AnT predictions based on temporal (Predictor 1) and anthropometric (Predictor 2) data.

From outputs of Predictors 1 and 2, a final prediction, composed of intermediate predictions of Predictors 1 and 2, is calculated. A final prediction is a result of the combination of rbₒᵤₜₚᵤₜ and mlpₒᵤₜₒᵤₜ descriptors:

$$AT / AnT = m ( rb ut , m l put ) , \qquad ( 11 )$$

where m refers to the aggregation function fed with rb [symbol] ut (regression-based prediction from Predictor 2) and ml [symbol] put (MLP prediction from Predictor 1), and AT and AnT are expressed in beats per minute (bpm).

Finally, based on a descriptor f, a pre-defined threshold tao to the range of tolerance values for each index is evaluated to ensure consistency to the final prediction to be displayed to users:

$${AT}_{tao} ( f ) = {AT}_{MLP} ( {f}^{′} ) + {AT}_{regbas} ( {f}^{″} ) / {n}_{estimators} \qquad ( 12 )$$

$${AnT}_{tao} ( f ) = {AnT}_{MLP} ( {f}^{′} ) + {AnT}_{regbas} ( {f}^{″} ) / {n}_{estimators} \qquad ( 13 )$$

where tao is related to the post-processed AT and AnT final predictions (following the rules defined for their predictions), MLP are the MLP-model predictions, regbas are the regression-based model predictions, nₑₛₜᵢₘₐₜₒᵣₛ is the number of models employed for AT and AnT predictions, f is a descriptor composed of temporal and anthropometric features, and f′, f″ are subsets of the whole set of features of f, containing anthropometric (f′) and temporal (f″) features, respectively.

Hence, the final AT/AnT predictions rely on a final combination module, which employs machine learning ensemble techniques to provide a more accurate AT/AnT estimation.

This module describes machine learning (ML) techniques for coping with overfitted or biased-fixed models, providing more realistic and accurate predictions. Among techniques employed:
I) Bagging: reduces the variance differences among models;
II) Boosting: deals with biases of models based on sharing their weights; and
III) Stacking: creates simpler models whose predictions feed more complex prediction architectures.

Another very important feature incorporated to AT/AnT prediction model is the VO2Max as input. The VO2Max indicator represents the maximum oxygen intake capability, and it is highly correlated to AT and AnT indexes. Considering the progression nature established by the relation AT<<AnT<<HRMax/VO2Max, the restriction implied by VO2Max as an input is necessary to ensure such relation.

### Data Preparation, Pre-Processing and Transformation

As presented above, several data preparation, pre-processing and transformation steps must be conducted to ensure the correct usage of data by the ML models. More specifically, the data must be gathered and prepared appropriately, including demographic and temporal features (columns) required for further evaluation. This first preparation comprises the collection of samples in a tabular scheme, described in an n-observation fashion. Then, the pre-processing and transforming steps depict the data changing and cleaning task, first to be in the pattern to feed the predictors employed (Predictor 1 for temporal data and Predictor 2 for anthropometric data), and then to transform into records that enable the prediction of AT and AnT.

The cleaning and pre-processing tasks are related to fixing inconsistencies, encoding data (calculating the window grid of n-temporal data), and standardization. Finally, the transforming step consists of intermediate processing steps, such as domain change and data encoding. The domain change maps labels or features used on the prediction models to a new learned value space. The data encoding finds a generalization of temporal data into a one-dimensional feature vector to feed the final AT/AnT predictor using, for instance, machine learning modules, such as LSTM.

All the intermediate processes between the first gathered data and the final prediction of AT and AnT are related to processing and transforming tasks, essential to provide the correct prediction of such fitness indexes.

In summary, the method for predicting aerobic threshold (AT) and anaerobic threshold (AnT) for establishing the optimal aerobic training zone (OATZ) comprises:
receiving personal data;
feeding a MLP machine learning model with the personal data to generate a first intermediate prediction mlpoutput;
capturing temporal signals (x) including heart rate and speed readings;
splitting temporal signals into n-second time windows;
processing the n-second time windows, wherein the processing includes:
   identifying points where heart rate and speed signals satisfy a stability condition;
   extracting features of stable temporal signals (xₜ) through an LSTM model;
   feeding a regression-based model with the features of stable temporal signals to generate a second intermediate prediction (rbₒᵤₜₚᵤₜ)
combining intermediate predictions performed by machine learning models and regression-based estimators, based on a pre-defined threshold tao to the range of tolerance values for each index;

$${AT}_{tao} ( f ) = {AT}_{MLP} ( {f}^{′} ) + {AT}_{regbas} ( {f}^{″} ) / {n}_{estimators}$$

$${AnT}_{tao} ( f ) = {AnT}_{MLP} ( {f}^{′} ) + {AnT}_{regbas} ( {f}^{″} ) / {n}_{estimators}$$

where tao is related to the post-processed AT and AnT final predictions, MLP are the MLP-model predictions, regbas are the regression-based model predictions, nₑₛₜᵢₘₐₜₒᵣₛ is the number of models employed for AT and AnT predictions, f is a descriptor composed of temporal and anthropometric features, and f′, f″ are subsets of the whole set of features of f, with the anthropometric (f′) and temporal features (f″) features, respectively;
defining the OATZ as the range [AT, AnT], in beats per minute and pace.

An alternative embodiment of the present invention comprises two main differences: (i) the application of a single-feature extraction module, without splitting personal and observed data performed, and (ii) instead of two models for the prediction task, only one would be required, considering the personal and measured data will compose a single feature vector to represent the data. This way, the feature evaluation is performed by a single predictor for estimating the best feature vectors according to its generalization performance, feeding a single prediction model that deals with one-encoded vector composed of personal and measures processed data.

Obviously, personal and observed data may present different prediction impacts over the defined models, and according to previous experiments, it is hard to deal with both feature natures to cope with the precise and accurate prediction of AT and AnT indexes. The event of applying more sophisticated and powerful models, such as deep neural networks, would change this scenario but would add extra computational cost as a drawback.

Hence, this alternative embodiment proposes a more compact pipeline to be followed in the AT/AnT prediction task, composed of a single feature extraction module fed by personal and observed data, and only one prediction model, considering all the features will feed the same prediction architecture. The main drawback related to such a solution includes a prediction module that comprises more complex architectures compared to the preferred embodiment.

The alternative embodiment is proposed to be applied to scenarios in which the computational cost is not a constraint, and also the storage of more complex models and its configurations (weights) do not compromise the setup and functionality of the proposed product or its placement into the target device.

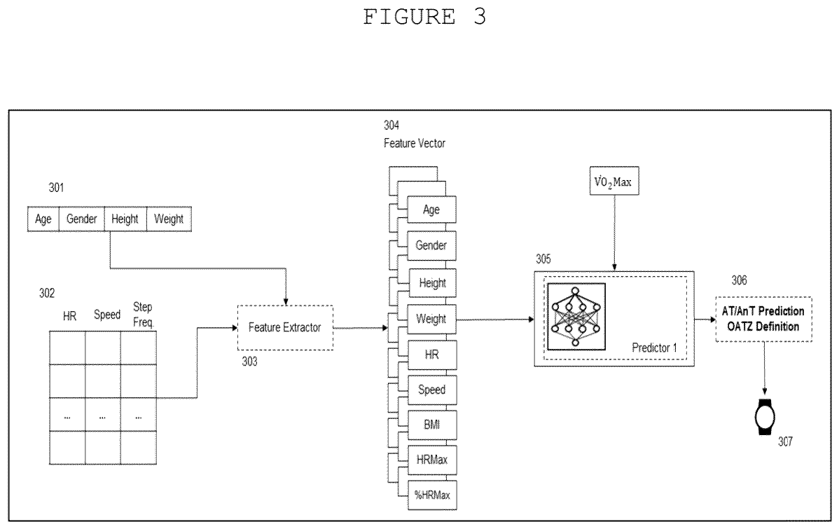

FIG. 3 presents a brief overview of the alternative embodiment. Firstly, information from user profile (301) is captured, including age (in years), gender (male/female), height (in centimeters) and weight (in kilograms). Secondly, temporal inputs (302) composed of readings of, for instance, heart rate, speed and step frequency, are collected during N seconds. Both profile and measured information (301 and 302, respectively) are processed by a feature extraction module (303). Finally, the processed data feed the prediction module (305), first designed as a deep neural network model.

The prediction of AT and AnT values, and further OATZ range (306) relies on the prediction module (305). Finally, with the OATZ information in the smartwatch (307), the intensity of a physical exercise can be monitored to implement training improvements and possible adaptations, focused on enhancing the fitness outcome from it, which results in important feedback for the efforts and habits carried out regularly.

Although the present invention focuses on smartwatches to process and show the AT/AnT/OATZ predictions, it can be extended to other wearable devices.

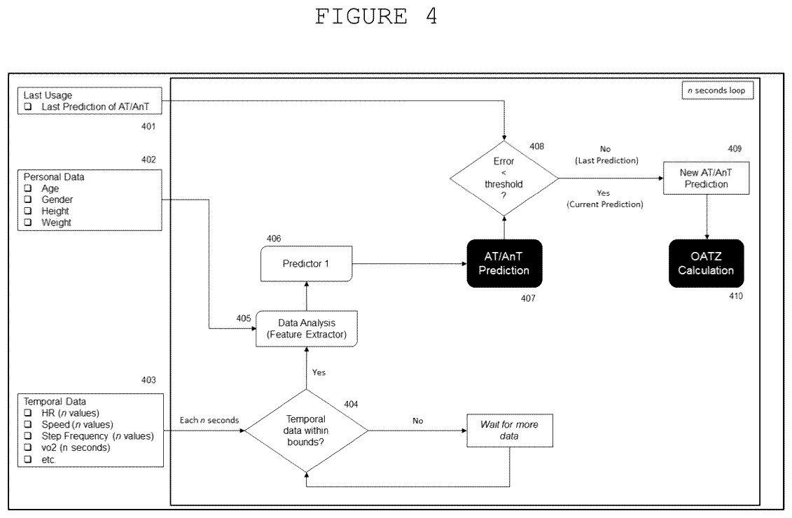

FIG. 4 presents the prediction pipeline of the alternative embodiment. In more details, information from user profile (402) is received, then the n-seconds temporal inputs (403) are captured and fed to the feature extractor module (405). The data analysis module (405) output is assigned to a machine-learning model (406) in order to predict the fitness measures (407). Finally, there is a post-processing step (408) to decide whether to output the new prediction (409) or to repeat the last stable prediction (401), depending on whether the feature vector deviation exceeds a predetermined threshold. The OATZ (410) is further displayed.

As an exemplary alternative embodiment, it is possible to infer that it could be used together with the Gait Enhancing & Motivating System (GEMS). GEMS is basically an integration of an exoskeleton, embedded software, virtual reality (VR) glasses, and a smartwatch or smartphone. Its objective is to customize training for the user. It has the capacity to increase load during exercising applying a counterforce during workout sessions. Utilizing AT and AnT predictions it is possible to control training intensity to keep the user inside the OATZ, achieving even better results. Creating a more personalized training routine.

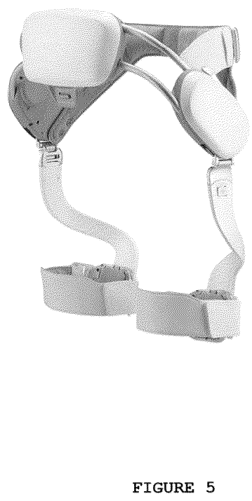

FIG. 5 shows the GEMS exoskeleton device. A lower body attachment focused on created a more immersive virtual training environment. It has actuator to apply counterforce during exercise routines and sensors to measure angle, balance and other features related to workout execution.

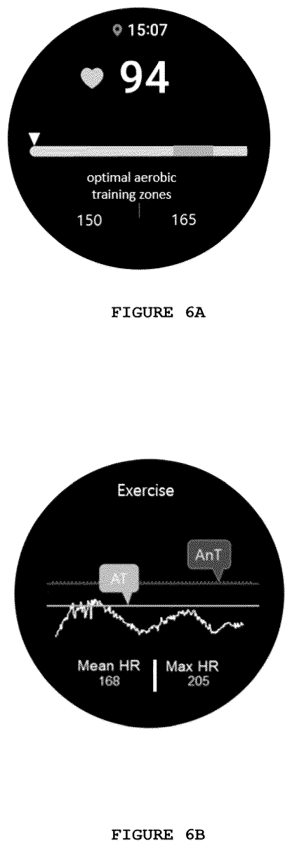

The benefits related to this solution concern the constant improvement of user training efficiency from supervised training. In practical ways, ordinary users can track their fitness conditions, adapt their training, and improve their outcomes by the feedback shown on the device, as depicted in FIG. 6.

Although the present invention has been described in connection with certain preferred embodiments, it should be understood that it is not intended to limit the disclosure to those particular embodiments. Rather, it is intended to cover all alternatives, modifications and equivalents possible within the spirit and scope of the disclosure as defined by the appended claims.

In response to the Notice to File Corrected Papers, the claims are presented herein to improve quality thereof:

1. A method of predicting aerobic threshold (AT) and anaerobic threshold (AnT) and establishing an optimal aerobic training zone (OATZ), the method comprising:
receiving personal data;
feeding a regression-based model with the personal data to generate a first intermediate prediction (rbₒᵤₜₚᵤₜ);
capturing temporal signals (x) including heart rate and speed readings;
splitting the temporal signals into n-second time windows;
processing the n-second time windows, wherein the processing includes:
identifying points where heart rate and speed signals satisfy a stability condition;
extracting features of stable temporal signals (xₜ) through a neural networks-based model;
feeding a MLP machine learning model with the features of stable temporal signals to generate a second intermediate prediction (mlpₒᵤₜₚᵤₜ)
combining intermediate predictions performed by machine learning models and regression-based estimators, based on a pre-defined threshold tao to a range of tolerance values for each index;
$${AT}_{tao} ( f ) = {AT}_{MLP} ( {f}^{′} ) + {AT}_{regbas} ( {f}^{″} ) / {n}_{estimators}$$
$${AnT}_{tao} ( f ) = {AnT}_{MLP} ( {f}^{′} ) + {AnT}_{regbas} ( {f}^{″} ) / {n}_{estimators}$$
where tao is related to post-processed AT and AnT final predictions, MLP refers to MLP-model predictions, regbas refers to regression-based model predictions, nₑₛₜᵢₘₐₜₒᵣₛ is a number of models employed for AT and AnT predictions, f is a descriptor composed of temporal and anthropometric features, and f′, f″ are subsets of a whole set of features of f, with anthropometric (f′) and temporal features (f″) features, respectively; and
defining the OATZ as a range [AT, AnT], in beats per minute and pace.

2. The method as in claim 1, wherein the personal data includes one or more of information of height, weight, gender and age registered by a user.

3. The method as in claim 1, wherein device's sensors register temporal data as heart rate and speed signals.

4. The method as in claim 1, wherein the stability condition for processing temporal observation from device's sensors comprises:
calculating a standard deviation of a temporal signal with a moving average X_{A->A+M}, which is an average of the temporal signal with a sliding window of M seconds with a stride of M seconds;
accumulating every stable 30 seconds of temporal data, excluding windows that do not satisfy stability criteria, building a new filtered temporal signal that comprises both temporal behavior and outlier-free information;
$${temp}_{signal} = {temp}_{signal} + ( {signal}_{data} \leq {StandardDeviation}_{Threshold} ) ,$$
where temp_{signal} corresponds to heart rate and speed filtered signals, signal_{data} is raw temporal data and respective StandardDeviation_{Threshold} relates to a corresponding threshold defined for a stable window criteria.

5. The method as in claim 1, wherein time-series features are extracted from temporal signals (xₜ) based on a Long Short-term Memory (LSTM):
$${f}_{t} = {σ}_{g} ( {W}_{f} \times {x}_{t} + {U}_{f} \times {h}_{t - 1} + {b}_{f} ) ;$$
$${i}_{t} = {σ}_{g} ( {W}_{i} \times {x}_{t} + {U}_{i} \times {h}_{t - 1} + {b}_{i} ) ;$$
$${o}_{t} = {σ}_{g} ( {W}_{o} \times {x}_{t} + {U}_{o} \times {h}_{t - 1} + {b}_{o} ) ;$$
$${c}_{t}^{′} = {σ}_{c} ( {W}_{c} \times {x}_{t} + {U}_{c} \times {h}_{t - 1} + {b}_{c} ) ;$$
$${c}_{t} = {f}_{t} \cdot {c}_{t - 1} + {i}_{t} \cdot {c}_{t}^{′} ;$$
$${h}_{t} = {o}_{t} \cdot {σ}_{c} ( {c}_{t} ) ;$$
$${x}_{t} = \frac{{\sum }_{i = 1}^{n} {x}_{{t}_{i}}}{n - 1} ;$$
$$C o V = \frac{{\overline{x}}_{t}}{\sqrt{\frac{{Σ}_{i = 1}^{n} {( {x}_{{t}_{i}} - {\overline{x}}_{t} )}^{2}}{n - 1}}} ;$$
where fₜ is a forget gate, iₜ is an input gate, oₜ is an output gate, cₜ is a cell state, hₜ is a hidden state, σ_{g} means sigmoid, and σ_{c} represents a tanh function, and CoV is variation coefficient of xₜ.

6. The method as in claim 5, wherein intermediate descriptor mlpₒᵤₜₚᵤₜ is calculated based on an MLP which includes i hidden layers with j neurons that has been trained with individuals who had reference measured AT and AnT.

7. The method as in claim 1, wherein personal features, extracted from user's anthropometric characteristics, provide rbₒᵤₜₚᵤₜ descriptor:
$${rb}_{output} = c + ( rb 0 * G ) + ( rb 1 * A ) + ( rb 2 * W ) + ( rb 3 * H )$$
where G refers to gender (0=female and 1=male), A refers to age in years, W refers to weight in Kg, H refers to height in cm, rb0 to rb3 and c referring to regression-based model parameters and bias, respectively, calculated during the training.

8. The method as in claim 1, wherein performing the prediction of aerobic/anaerobic threshold, performed in AT/AnT Prediction module, comprises:
$$AT / AnT = m ( rb ut , m l put )$$
where m refers to the mean function fed with rb [symbol] ut and ml [symbol] put, and AT and AnT are expressed in beats per minute (bpm).
